(19) United States
(12) Patent Application Publication
(10) Pub. No.: US 2010/0280405 A1
(43) Pub. Date: Nov. 4, 2010

(54) Method For Testing Visual Field

(75) Inventors: Tomasz MUSIALIK, Zawiercie (PL); Konrad STEPIEN, Tychy (PL)

Correspondence Address: WILLIAM J. SAPONE;COLEMAN SUDOL SAPONE P.C., 714 COLORADO AVENUE, BRIDGE PORT, CT, 06605, US

(73) Assignee: OPTOPOL TECHNOLOGY S.A., Zawiercie (PL)

(21) Appl. No.: 12/769,711

(22) Filed: Apr. 29, 2010

(30) Foreign Application Priority Data
Apr. 30, 2009 (PL) P.387923

(51) Int. Cl. A61B 5/00

(52) U.S. Cl. 600/558

(57) ABSTRACT

The invention relates to a method for testing visual field of a patient with use of a computer system (7) connected to an output-matrix of light points (6) having controlled brightness and to an input device (4) for measurement of patient's reaction. The method comprises the steps of: a) selecting by the computer system (7) the subset of light points from the set comprising all not yet examined light points of the matrix (6), b) defining by the computer system (7) the initial brightness for each point selected in step a), c) projecting by the computer system (7) subsequent light points of the matrix (6) from the subset of light points selected in step a) with different levels of the brightness starting from the initial brightness determined in step b), registering the reaction of the patient indicating whether the projected point is seen by him by the computer system (7) by means of the input device (4), and defining by the computer system (7) the level of the sensitivity of the retina of the patient in the given point, based on registered reactions of the patient, d) defining by the computer system (7) the map of the sensitivity of the retina of the patient. In order to enable shortening of the duration of the test and decreasing its arduousness for a patient as well as to enable to stop the examination before examining all points of the dot matrix without the loss of previously registered information on the quality of the patient's vision, before step d) at least one cycle of steps a)-c) is carried out, wherein in the first cycle of steps a)-c) the initial brightness of a given point projected is defined in step b) at least on the basis of the data concerning the patient, and in the second and each successive cycle of steps a)-c) the initial brightness of a given point projected is defined in step b) on the basis of the brightness determined for the other light points of the matrix (6).

### TECHNICAL FIELD

The invention relates to a method for testing visual field of a patient with use of a computer system connected to an output-matrix of light points having controlled brightness and to an input device for measurement of patient's reaction.

### BACKGROUND

Patent specification U.S. Pat. No. 4,927,259 discloses an apparatus and method for testing the field of vision of the patient. The apparatus comprises light point sources distributed over the visual field of the patient, an input device for accepting the patient's responses and a control unit controlling the projection of light points. For every point the patient gives the answer “seen” or “not seen” which are relayed to the control unit, which based on it increases the brightness of the point if the patient does not see it or decreases the brightness of the point if the patient sees it, whereas this change has a logarithmic character, and the method comprises at least one phase of the inverted brightness in case of the change of trend (from “seen” on “not seen” or vice-versa).

International patent publication WO 9424925 discloses a method of determining threshold value of patient's perception of visual stimuli for every chosen test-series in which there is used an estimated error of the calculation of the threshold value in this test-series for determining whether the series has to be continued or not. This allows stopping the test-series whenever the threshold value of the given series can be determined with the sufficient accuracy. Preferably, a posterior probability function is used for estimating the error. An apparatus for carrying out the method includes a control unit operating according to this method.

Other devices and methods of testing visual field are also disclosed in documents U.S. Pat. No. 4,558,933, U.S. Pat. No. 4,349,250, U.S. Pat. No. 4,334,738, EP 1442695, GB 2305515.

### SUMMARY OF THE INVENTION

It is an object of the present invention to provide a method for testing the visual field of the patient, which would be simple in implementation, enable for the considerable shortening of time of the examination and decreasing its arduousness for the patient, and additionally make it possible to stop the examination in any moment before testing all points of the matrix and to generate the result based on information registered up to that moment.

The invention relates to the method of testing the visual field of a patient which comprises steps of: a) selecting by the computer system the subset of light points from the set comprising all not yet examined light points of the matrix, b) defining by the computer system the initial brightness for each point selected in step a), c) projecting by the computer system subsequent light points of the matrix from the subset of light points selected in step a) with different levels of the brightness starting from the initial brightness determined in step b), registering the reaction of the patient indicating whether the projected point is seen by him by the computer system by means of the input device, and defining by the computer system the level of the sensitivity of the retina of the patient in the given point, based on registered reactions of the patient, d) defining by the computer system the map of the sensitivity of the retina of the patient. This method is characterized in that before step d) at least one cycle of steps a)-c) is carried out, wherein in the first cycle of steps a)-c) the initial brightness of a given point projected is defined in step b) at least on the basis of the data concerning the patient, and in the second and each successive cycle of steps a)-c) the initial brightness of a given point projected is defined in step b) on the basis of the brightness determined for the other light points of the matrix.

Said data of the patient in the first cycle of steps a)-c) advantageously comprise at least the age of the patient and optionally may comprise the sex and/or the ethnic group, however in the second and each following cycle of steps a)-c) the initial brightness of a given point projected is defined in step b) preferably on the basis of the brightness determined previously for points in the vicinity of the given point or based on approximation or interpolation of the data set containing the levels of brightness determined previously for the other light points of the matrix.

Determining the initial value of the level of the brightness of light points significantly decreases the number of projections of a given point and a corresponding number of measurements of patient's reaction. In the first step, the level of the brightness is determined for example on the basis of the average results of the sensitivity of the retina of patients from a given age range, whereas in second and successive cycles of the test the results obtained in previous cycles are used. Owing to this, even if the test will be interrupted by the patient, the computer system can determine the map of the sensitivity of the retina of the patient on the basis of the information for the other points of the matrix, comprising both registered reactions of the patient, as well as estimated values.

### BRIEF DESCRIPTION OF THE DRAWINGS

These and the other advantages of the invention are presented below on the basis of preferred examples of its implementation and with reference to the drawings, of which

FIG. 1 schematically shows a computer static perimeter which can be used for implementation of a method according to the invention;

FIG. 2 shows an algorithm illustrating steps of the first example of implementation of an exemplary method according to the invention;

FIG. 3 shows an algorithm illustrating steps of the other exemplary method according to the invention;

FIG. 4 shows in detail the algorithm of implementation of the second and following steps of the test of the exemplary method according to the invention as presented in FIG. 3; and

FIGS. 5a-5d show exemplary subsets of points tested in successive cycles of testing the sensitivity of retina.

### DETAILED DESCRIPTION OF THE INVENTION

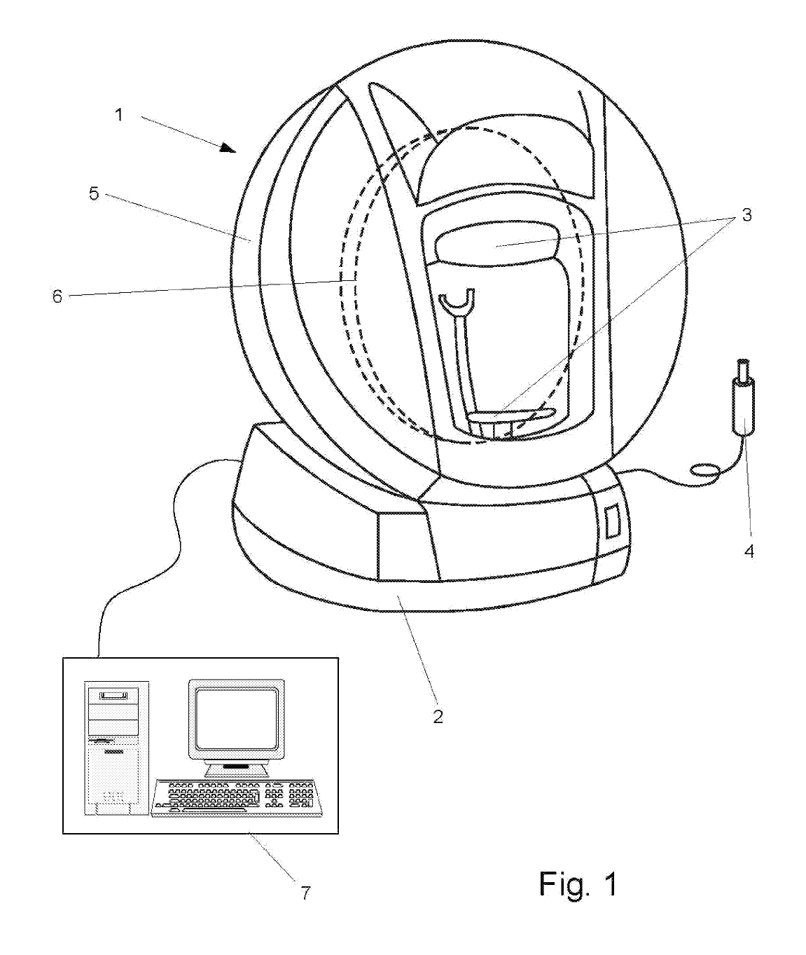

FIG. 1 schematically illustrates an exemplary static perimeter 1 for testing the visual field, which can be employed in a method according to the invention. The perimeter 1 comprises a housing 2, a device 3 for supporting a chin and a forehead of a patient, a communication button 4, a perimeter bowl 5 integrated with the housing 2 and containing a matrix of light points 6 and a computer system 7 connected to the communication button 4 and the perimeter bowl 5.

During the examination the patient is seated before the perimeter 1 having one eye covered and the head immobiled in the device 3. The patient looks with his or her second eye into the perimeter bowl 5 on which the light points 6 are shown successively, wherein the selection and the brightness of a given light point 6 which is shown in a given moment are controlled by the computer system 7. The task of the patient is to signalize by means of the communication button 4 whether the point is seen or is not seen. The patient's answers have therefore a binary character (YES/NO).

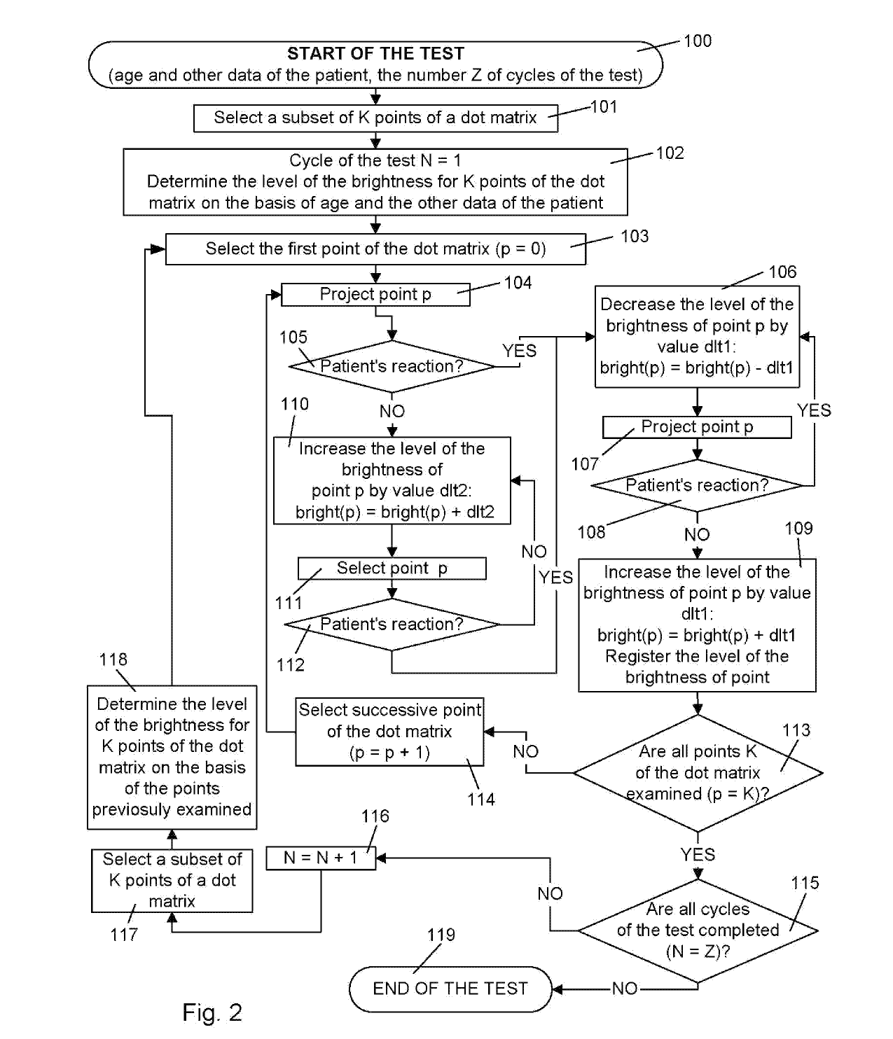

FIG. 2 presents particular steps of an exemplary method for testing the visual field according to the invention with the use of the exemplary static perimeter 1 of FIG. 1.

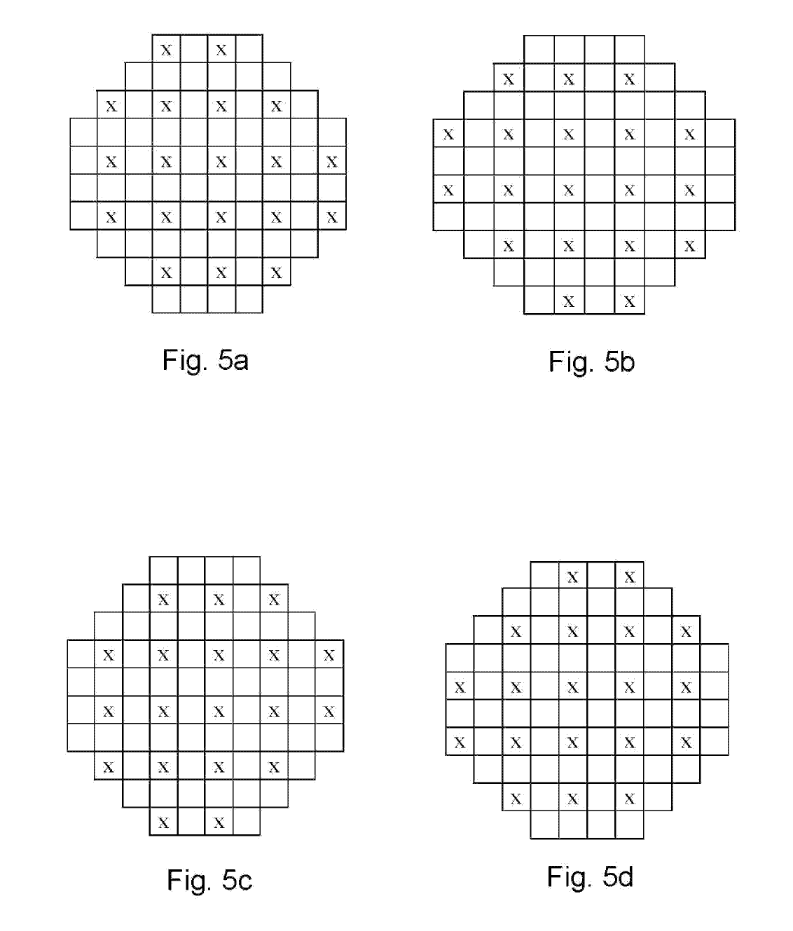

In the first step 100, the number Z (e.g. 4) of cycles of the test is determined and the age and optionally other data of the examined patient are introduced to the computer system. The first cycle of the test (101) begins with selecting the subset of K points from the group of light points 6 of the perimeter bowl 5 which will be shown to the patient. These points should be evenly distributed over the perimeter bowl 5, creating for example a dot matrix shown in FIG. 5a. The initial brightness for particular points of this subset is determined at least on the basis of the age of a patient (102) what shall be discussed in the further part of the description. Subsequently, the computer system 7 selects in step 103 the first point of the dot matrix, projects this point to the patient (104) and the patient signalizes by means of the communication button 4 whether the point (105) is seen. If so, in step 106 the brightness of the point is decreased by a given predefined value dlt1 and after projecting it again (107) the reaction of the patient is checked in step 108. Steps 106-108 are repeated as long as the point is seen, then again the brightness of the point is increased by a given predefined value dlt1 (109) and such a resulted value is assumed as the measured level of the sensitivity of the retina of the patient in this point.

If in step 105 the patient signalizes that a given light point is not seen by him the brightness of this point is increased (110) by a given predefined value dlt2, the point is projected again (111) and the reaction is checked again in step 112. Steps 110-112 are repeated as long as the point is not seen by the patient, then the cycle of steps 106-108 is carried out again, and eventually the measured level of the sensitivity of the retina of the patient in the given point is determined in step 109 as the brightness of this point increased by the given predefined value dlt1.

The above procedure is carried out for all points of the selected subset of light points (113, 114), then, unless all cycles of the test have yet been carried out (115), the successive cycle of the test begins (116) with selecting the next subset of light points 6 of the perimeter bowl 5, which will be shown to the patient (117), for example a subset forming one of dot matrixes shown in FIG. 5 b-c (for the next three cycles of the test), wherein dot matrixes for particular cycles do not overlap with each other and their light points are evenly distributed on the perimeter bowl. Advantageously subsets of light points in successive cycles of the test have an equal configuration but are dislocated spatially, for example by one point in relation to each other.

In the second and successive cycles of the test according to the present invention, the computer system 7 however determines in step 118 the initial brightness of points of the currently selected dot matrix on the basis of the results of sensitivity recorded in steps 109 for the points in the vicinity of the given point and examined in previous cycles. In calculations carried out in this step, the results for points directly adjacent to the given point of the current dot matrix, results for points located in a predetermined neighbourhood of the given point or also all examined points can be taken into account. In the simplest case, determination of the initial level of the brightness consists in calculating an arithmetic average of the results received for points from given neighbourhood of the given point. In more sophisticated implementations, the calculations may comprise calculation of a weighted average, the weights of which will be inversely proportional to the distance of the given point from other neighbouring points for which the level of the sensitivity has already been measured, or any other algorithm of interpolation or two-dimensional approximation, such as for example radial basis function interpolation.

After defining the initial values of the brightness (118) for the successive dot matrixes, the computer system performs step 103 and subsequently performs the successive cycle of steps 104-109 in the result of which the measured levels of the sensitivity for the consecutive subset of points of the dot matrix are successively being registered.

After completing a given number Z of cycles, the computer system 7 ends up the test (119).

In the preferred case, light points of the perimeter bowl are divided into four subsets and four cycles of the test are carried out. Exemplary subsets of the matrix examined in the successive cycles are shown in FIGS. 5a-5d.

In the practical implementation it is advantageous that the step of increasing of the brightness dlt2 is two-fold greater than the step of decreasing of the brightness dlt1. It is especially advantageous if dlt1 amounts −3 dB while dlt2 amounts −6 dB.

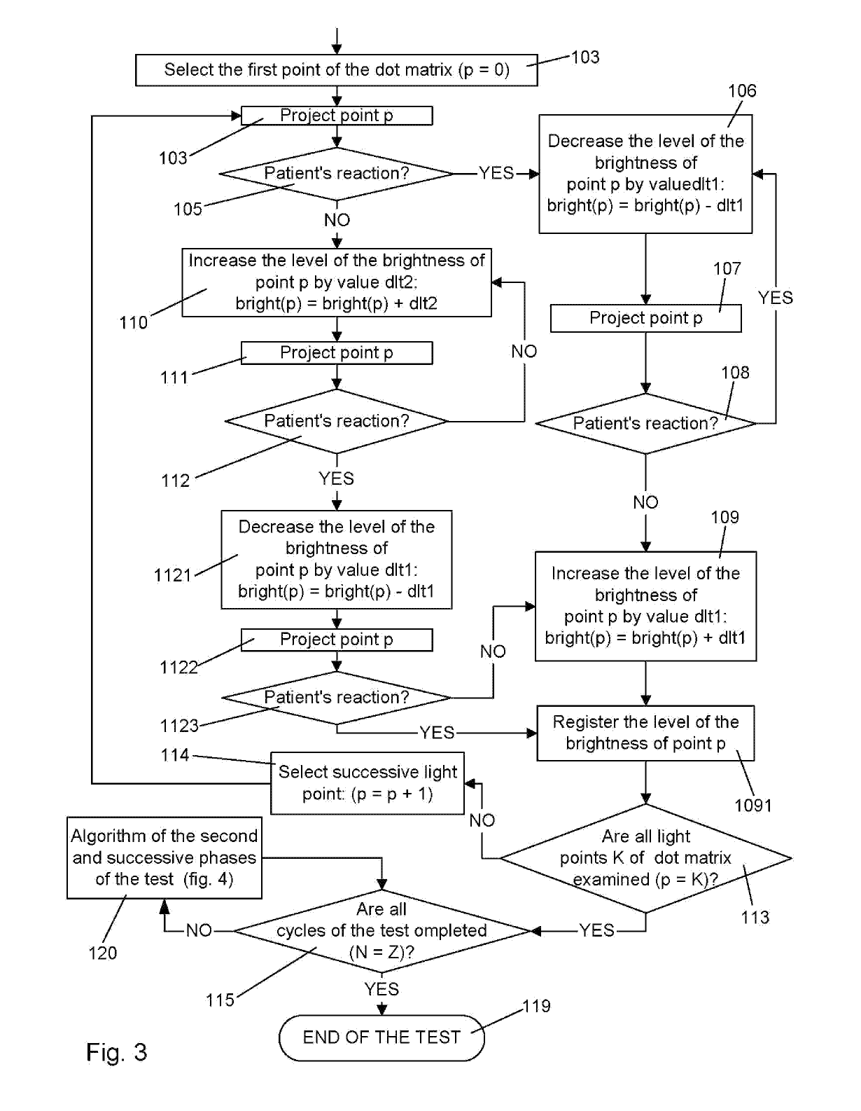

Owing to the above mentioned mutual relation between the values of steps dlt1 and step dlt2, it is possible to modify the path of steps of the sensitivity test for a given point, which in FIG. 2 comprises steps 105-109, with a path leading to an algorithm shown in FIG. 3.

This modification consists in defining two separate operation paths: the first one for the case when the patient has seen the point of initial brightness and the second one for the case when the patient has not seen the point of initial brightness. It is attained by removing the passage connection from step 112 to step 106 and introducing three additional steps comprising: step 1121 of projecting the point with brightness decreased by −3 dB if in step 112 the patient has seen the point; step 1122 comprising projection of the point of the brightness determined in step 1121; and then step 1123 of determining whether the patient has seen the point projected in step 1122. In the case when in step 1123 the patient does not see the point, the sensitivity for the point is recorded (1091) as the last brightness value for which the patient had seen the point in step 112. However if in step 1123 the patient sees the point then the current brightness (1121) is directly recorded as the value of the sensitivity (1091).

As compared with the algorithm from FIG. 2, such modification enables reduction by one the number of projections of tested point what in case of testing several dozen of points of a retina should be considered as considerable improvement.

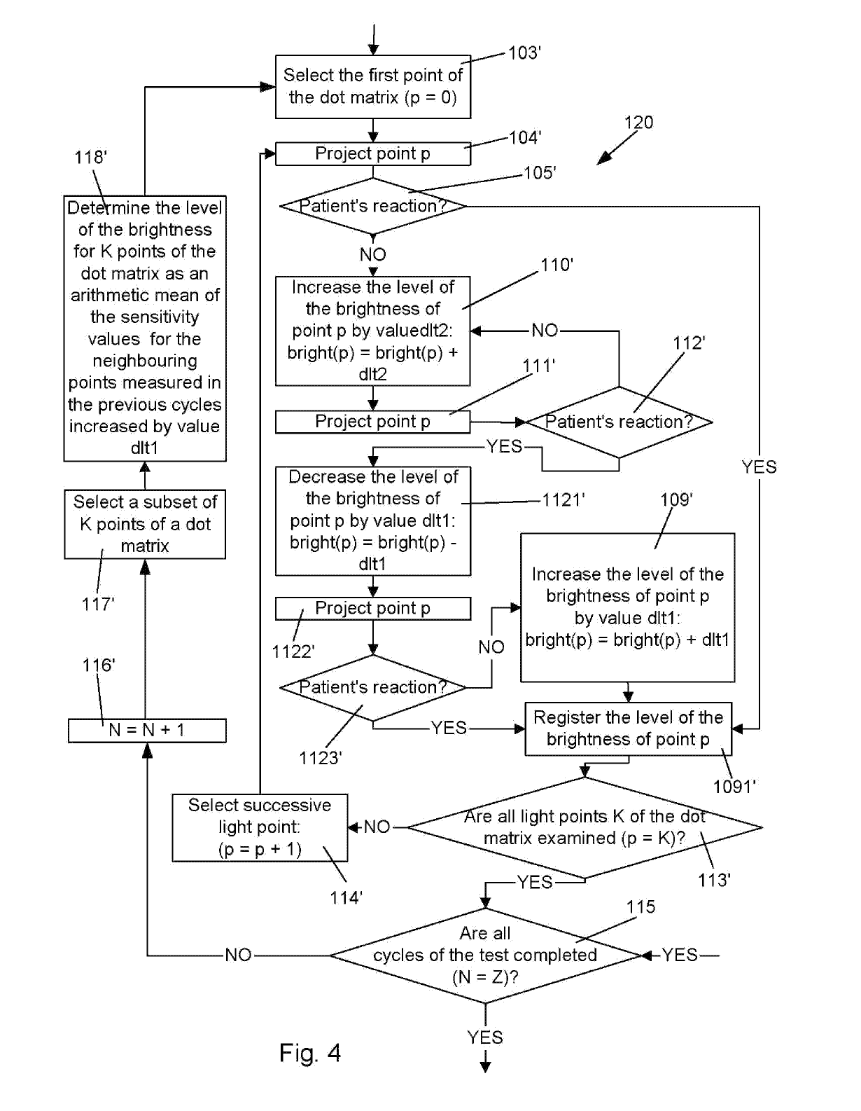

Additionally in the second and consecutive cycles of the test 120, as illustrated on the basis of an algorithm from FIG. 4, except for the described above separating operation paths, the path of determining the sensitivity of the point was additionally simplified for the case when the patient has seen the point of the initial brightness. The brightness in these cycles is determined as described above on the basis of the sensitivity of the neighbouring points from the previous cycles, and preferably as an arithmetic average of the neighbouring points levels of the sensitivity. From the original version of this path (steps 105-109 in FIG. 2) the loop 106-108 was removed. Thus in case when the patient sees the point of the initial brightness (105′) then this value is directly assumed as the sensitivity in this point in step 1091′.

The algorithm shown in FIGS. 3 and 4 is a simplification of the algorithm from FIG. 2. While in the algorithm from the FIG. 2, the sensitivity of each point in any cycle of the test is defined by a threshold value method (i.e. a method enabling for determining sensitivity lower than as well as sensitivity higher than the initial value of the brightness), in the algorithm shown in FIGS. 3 and 4 a threshold value algorithm (modified insignificantly in relation to the threshold value algorithm from FIG. 2) is performed only in the first cycle of the test whereas in the second and the successive cycles the sensitivity in a given point is determined using a suprathreshold value method enabling for determining the sensitivity for the brightness higher than the initial value.

In FIG. 5 exemplary subsets of exemplary dot matrixes of light points are shown. In a case of performing test comprising four cycles such a dot matrix can be preliminarily divided into four separate subsets of points as shown in FIG. 5a-5d which in the aggregate cover the whole dot matrix. Alternatively, as described above, according to the invention it is also possible to randomly select points for each cycle of a test.

The skilled technician of the field of the invention will easily notice that each one of above described algorithms can be interrupted practically in any moment, for example at the request of the patient, even before completing the initially defined number of cycles of the test. In such a situation, the computer system 7 will calculate the values of the sensitivity in the unexamined light points of the matrix 6 based on recorded results of the sensitivity for the points examined in previous cycles of the test. These calculations can be carried out in a similar manner as in step 118 or 118′ based on an arithmetic average or a weighed average of the results measured and/or determined on the basis of the age and other data of the patient for the points from a given neighbourhood of the given point, or any other algorithm of the interpolation or two-dimensional approximation of these results.

The presented examples of the implementation should not be treated as exhaustive; some details of the drawings were shown only schematically, they may not preserve the scale and certain details could be enlarged or diminished for the purpose of better illustration of the invention the gist of which is characterized in patent claims.

1. A method for testing visual field of a patient with use of a computer system (7) connected to an output-matrix of light points (6) having controlled brightness and to an input device (4) for measurement of patient reaction, comprising the steps of:
a) selecting by the computer system (7) the subset of light points from the set comprising all not yet examined light points of the matrix (6),
b) defining by the computer system (7) the initial brightness for each point selected in step a),
c) projecting by the computer system (7) subsequent light points of the matrix (6) from the subset of light points selected in step a) with different levels of the brightness starting from the initial brightness determined in step b), registering the reaction of the patient indicating whether the projected point is seen by him by the computer system (7) by means of the input device (4), and defining by the computer system (7) the level of the sensitivity of the retina of the patient in the given point, based on registered reactions of the patient,
d) defining by the computer system (7) the map of the sensitivity of the retina of the patient, characterised in that
before step d) at least one cycle of steps a)-c) is carried out,
wherein in the first cycle of steps a)-c) the initial brightness of a given point projected is defined in step b) at least on the basis of the data concerning the patient, and
in the second and each successive cycle of steps a)-c) the initial brightness of a given point projected is defined in step b) on the basis of the brightness determined for the other light points of the matrix (6).

2. The method according to claim 1, characterised in that said data concerning the patient comprise at least the age of the patient and optionally the sex and/or the ethnic group.

3. The method according to claim 1, characterised in that in said second and each successive cycle of steps a)-c) the initial brightness of a given point projected is defined in step b) on the basis of the brightness determined previously for light points of the matrix (6) in the vicinity of a given point.

4. The method according to claim 1, characterised in that in said second and each successive cycle of steps a)-c) the initial brightness of a given point projected is defined in step b) on the basis of approximation or interpolation of a data set comprising levels of brightness determined previously for the other light points of the matrix (6).

5. The method according to claim 1, characterised in that step c) comprises, for each point of the subset from step a) the steps of, determining by the input device (4) whether the patient sees the projected point or does not see the projected point, and alternate decreasing by the computer system (7) the brightness of this point by a given predefined value dlt1 in case the patient has seen the point or increasing the brightness of this point by a given predefined value dlt2, and projecting this point of changed brightness; and subsequently determining by the computer system (7) the level of the sensitivity of the retina of the patient in the given point, based on registered reactions of the patient.

6. The method according to claim 5, characterised in that said determining by the computer system (7) the level of the sensitivity of the retina of the patient in a given point, based on registered reactions of the patient, is performed in the moment when after decreasing the brightness, the patient does not see a given point or in the moment when the patient has seen the point of the initial brightness.

7. The method according to claim 6, characterised in that the level of the sensitivity of the eye of the patient in a given point is the last value of the brightness for which the patient has seen the given point.

8. The method according to claim 1, characterised in that in the second and each successive cycle of steps a)-c) the initial brightness of a given point projected is defined as an arithmetic average of the levels of the sensitivity of the retina of the patient determined by the computer system (7) for points in the vicinity of the given point, wherein the average is preferably increased by the value of dlt2.

9. The method according to claim 5, characterised in that the value dlt2 is two-fold greater than the value dlt1, whereas the value dlt1 preferably amounts −3 dB.

10. The method according to claim 1, characterised in that step a) comprises random selection of a given number of points.

11. The method according to claim 2, characterised in that in said second and each successive cycle of steps a)-c) the initial brightness of a given point projected is defined in step b) on the basis of the brightness determined previously for light points of the matrix (6) in the vicinity of a given point.

12. The method according to claim 2, characterised in that in said second and each successive cycle of steps a)-c) the initial brightness of a given point projected is defined in step b) on the basis of approximation or interpolation of a data set comprising levels of brightness determined previously for the other light points of the matrix (6).

13. The method according to claim 5, characterised in that in the second and each successive cycle of steps a)-c) the initial brightness of a given point projected is defined as an arithmetic average of the levels of the sensitivity of the retina of the patient determined by the computer system (7) for points in the vicinity of the given point, wherein the average is preferably increased by the value of dlt2.

14. The method according to claim 8, characterised in that the value dlt2 is two-fold greater than the value dlt1, whereas the value dlt1 preferably amounts −3 dB.

15. The method according to claim 2, characterised in that step c) comprises, for each point of the subset from step a) the steps of, determining by the input device (4) whether the patient sees the projected point or does not see the projected point, and alternate decreasing by the computer system (7) the brightness of this point by a given predefined value dlt1 in case the patient has seen the point or increasing the brightness of this point by a given predefined value dlt2, and projecting this point of changed brightness; and subsequently determining by the computer system (7) the level of the sensitivity of the retina of the patient in the given point, based on registered reactions of the patient.

16. The method according to claim 3, characterised in that step c) comprises, for each point of the subset from step a) the steps of, determining by the input device (4) whether the patient sees the projected point or does not see the projected point, and alternate decreasing by the computer system (7) the brightness of this point by a given predefined value dlt1 in case the patient has seen the point or increasing the brightness of this point by a given predefined value dlt2, and projecting this point of changed brightness; and subsequently determining by the computer system (7) the level of the sensitivity of the retina of the patient in the given point, based on registered reactions of the patient.

17. The method according to claim 4, characterised in that step c) comprises, for each point of the subset from step a) the steps of, determining by the input device (4) whether the patient sees the projected point or does not see the projected point, and alternate decreasing by the computer system (7) the brightness of this point by a given predefined value dlt1 in case the patient has seen the point or increasing the brightness of this point by a given predefined value dlt2, and projecting this point of changed brightness; and subsequently determining by the computer system (7) the level of the sensitivity of the retina of the patient in the given point, based on registered reactions of the patient.

18. The method according to claim 15, characterised in that the value dlt2 is two-fold greater than the value dlt1, whereas the value dlt1 preferably amounts −3 dB.

19. The method according to claim 16, characterised in that the value dlt2 is two-fold greater than the value dlt1, whereas the value dlt1 preferably amounts −3 dB.

20. The method according to claim 17, characterised in that the value dlt2 is two-fold greater than the value dlt1, whereas the value dlt1 preferably amounts −3 dB.
